(19) United States
(12) Patent Application Publication
(10) Pub. No.: US 2025/0121205 A1
(43) Pub. Date: Apr. 17, 2025

(54) SYSTEMS AND METHODS OF TREATING MEDICAL INDICATIONS BY ADMINISTERING DOSAGES OF LIGHT AND SOUND

(71) Applicant: INTO Technologies Inc., San Francisco, CA (US)

(72) Inventors: Stephen Auger, Santa Fe, NM (US); Jeffrey Bova, West Hollywood, CA (US); Benjamin Smarr, Carlsbad, CA (US); William Meeker, San Francisco, CA (US)

(21) Appl. No.: 18/912,252

(22) Filed: Oct. 10, 2024

Related U.S. Application Data
(60) Provisional application No. 63/543,701, filed on Oct. 11, 2023.

(51) Int. Cl. A61N 5/06

(52) U.S. Cl. CPC A61N 5/06; A61N 2005/0626; A61N 2005/0648; A61N 2005/0652; A61N 2005/0659; A61N 2005/0661; A61N 2005/0663

(57) ABSTRACT

A system and method for treating various medical indications is disclosed. Specific dosages of light are delivered to a subject. The dosages may be defined according to various parameters, including light wavelength, pulse frequency, intensity, area within the subject's field of vision, duration, pulse waveform shape, or a combination thereof. The dosages of light may be administered while the subject's eyes are closed. Auditory stimulus may also be provided, in synchronization with the administered dosages of light.

### CROSS-REFERENCE TO RELATED APPLICATIONS

This application claims priority to U.S. Provisional Patent Application No. 63/543,701, filed Oct. 11, 2023, the entirety of which is incorporated by reference herein. This application also incorporates by reference U.S. Provisional Patent Application No. 62/877,602, filed Jul. 23, 2019, U.S. Provisional Patent Application No. 62/961,435, filed Jan. 15, 2020, U.S. Provisional Patent Application No. 63/049,203, filed Jul. 8, 2020, U.S. Non-Provisional patent application Ser. No. 16/937,124, filed Jul. 23, 2020, International Application No. PCT/US20/43324, filed Jul. 23, 2020, U.S. Provisional Patent Application No. 63/171,900, filed Apr. 6, 2021, U.S. Non-Provisional patent application Ser. No. 17/714,756, filed Apr. 6, 2022, and International Application No. PCT/US22/23711, filed Apr. 6, 2022, in their entireties.

### TECHNICAL FIELD

The present disclosure relates to systems and methods for treating medical indications (including improved human functions) by administering a dosing of light.

### BACKGROUND

Pharmaceuticals are a customary solution to treating numerous medical indications. However, there is a need for a different approach to treating these medical indications as an alternative or a supplement to pharmaceuticals.

### BRIEF SUMMARY

Disclosed are systems and methods of treating medical indications by administering dosages of light. Also disclosed are systems and methods of treating medical indications by administering dosages of light in combination with administered and synchronized auditory stimulus.

### BRIEF DESCRIPTION OF DRAWINGS

The accompanying drawings, which are included to provide a further understanding of the invention and are incorporated in and constitute a part of this specification, illustrate preferred embodiments of the invention and together with the detailed description serve to explain the principles of the invention. In the drawings:

FIG. 1 illustrates a system, according to an embodiment.

FIGS. 2A and 2B illustrate examples of an arrangement of light emitters, according to an embodiment.

FIGS. 3A and 3B each illustrate an exemplary flow chart for treating a medical indication, according to an embodiment.

FIG. 4 illustrates an example computer system.

FIG. 5 illustrates an interface that may be used with the invention.

FIG. 6 illustrates an interface that may be used with the invention.

FIG. 7 illustrates stimulation frequencies plotted on a logarithmic frequency scale for different session lengths, where part (a) corresponds to a 5.5-minute session, part (b) corresponds to a 11-minute session, and part (c) corresponds to a 22-minute session.

FIG. 8 illustrates F-statistic ANOVA topographies, according to an embodiment.

FIG. 9 illustrates post-hoc comparisons of correlation coefficients (r) from the inventors' study.

FIG. 10 illustrates a photic driving effect from the inventors' study, where part (a) is a photic driving response from one study participant and part (b) is a channel-averaged spectrogram.

FIG. 11 is a table indicating the distribution of participants among the groups in the inventors' study.

### DETAILED DESCRIPTION

The present disclosure relates to systems and methods of treating medical indications by administering dosages of light, including in combination with auditory and/or other stimuli to a subject. Exemplary devices/systems are described herein, and additional devices/systems that may be used in combination with the methods described herein are described in U.S. Provisional Patent Application No. 62/877,602, filed Jul. 23, 2019, U.S. Provisional Patent Application No. 62/961,435, filed Jan. 15, 2020, U.S. Provisional Application No. 63/049,203, filed Jul. 8, 2020, U.S. Non-Provisional patent application Ser. No. 16/937,124, filed Jul. 23, 2020, and International Application No. PCT/US20/43324, filed Jul. 23, 2020, each of which are hereby incorporated by reference in their entireties, and are also described herein. While specific exemplary devices/systems are described or incorporated by reference herein, it should be understood that this is done for illustration purposes only. Other components and configurations may be used without departing from the spirit and scope of the invention.

The systems and methods of the present invention may be employed to treat various medical indications by administering one or more dosage(s) of light to a user. The dosage(s) of light may be administered to the user's eyes, so as to stimulate the user's retinal ganglion cells within the user's eyes. The dosage(s) of light may be administered while the user's eyes are closed, by transmitting the light dosage(s) through the user's eyelids. The light dosage(s) may be defined according to various parameters including wavelength, area within the user's field of vision, intensity, pulse frequency, duration, pulse waveform shape, photon quantity, or any combination thereof.

For context, stroboscopic or “flicker” stimulation is a form of periodic visual stimulation which induces geometric hallucinations through closed eyelids. While the visual effects of this form of sensory stimulation have received considerable attention, few studies have investigated the neural entrainment effects of periodic visual stimulation.

The systems and methods of the present invention may be employed to apply flicker stimulation to treat various medical conditions, including flicker stimulation that involves applying multimodal stimulation composed of two simultaneous strobe frequencies paired binaural beats which provided auditory stimulation at roughly the same frequency as the slower strobe. As explained in further detail below, the inventors conducted a study comparing this condition to sham stimulation where both strobes were set to very low frequencies with much weaker visual effects and in which the binaural beats were absent.

Additionally, the inventors compared both conditions to a control group in which participants focused on their breathing during eyes-closed meditation (no stimulation). The results demonstrate powerful evidence of neural entrainment at the frequency of the slower strobe in the experimental condition. Based on the inventors' findings, the present invention, provides benefits towards treating medical indications, such as potential therapeutic technique in psychiatric disorders. As another example, the present invention may, by applying a higher quantity of blue light with a specific flicker frequency, provide a beneficial effect for PTSD patients. The system of the present invention allows for sufficient flexibility and control of the dosing of stimulus/stimuli so as to provide versatility in treating various medical indications.

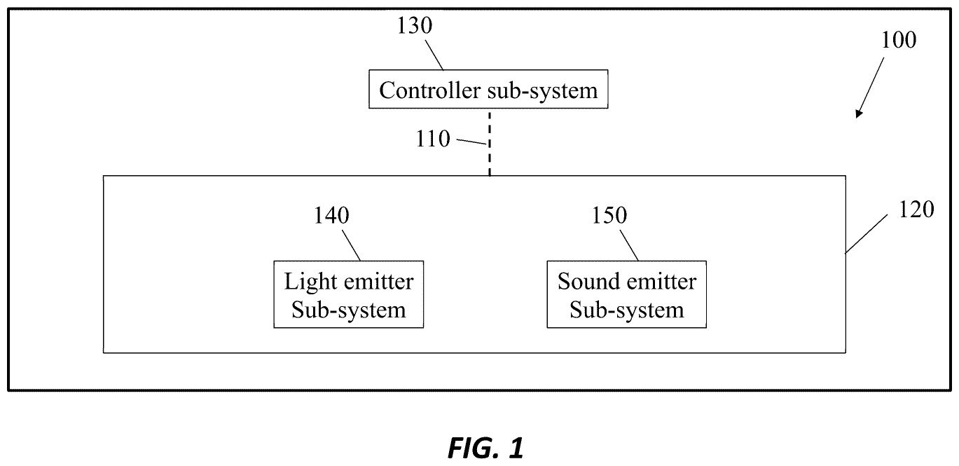

FIG. 1 illustrates a system 100 according to an example embodiment, which may be used in combination with the methods of treating medical indications disclosed herein. In one embodiment, the system 100 is configured to emit a light and/or sound stimulus to a user. According to an embodiment, the system 100 may include an emitter sub-system 120 and a controller sub-system 130. As explained in more detail below, the system 100 may be configured to apply light and/or sound stimulus to the user via the emitter sub-system 120, based on control by the controller sub-system 130.

The emitter sub-system 120 may include one or more stimulus emitters. For example, the emitter sub-system 120 may include a light emitter sub-system 140 to emit a light stimulus, and a sound emitter sub-system 150 to emit an auditory stimulus. These sub-systems as explained in detail below. In one embodiment, the light emitter sub-system 140 may be configured to apply one or more predetermined dosages of light to the user, to stimulate the user's retinal ganglion cells within the user's eyes. The predetermined dosages of light, for example, may be defined according to various parameters including:
(i) one or more predetermined wavelengths of light,
(ii) one or more predetermined areas/zones within the user's field of vision to direct the emitted light, including one or more individual areas/zones for each predetermined wavelength,
(iii) one or more predetermined intensities or amplitudes of light, including one or more individual intensities for each predetermined wavelength and/or area/zone,
(iv) one or more predetermined pulse frequencies or pulse rates (Hz), including one or more individual pulse frequencies for each predetermined wavelength, area/zone, and/or intensity,
(v) one or more durations to emit each wavelength, area/zone, intensity, and/or pulse frequency of light,
(vi) pulse waveform shape(s) (e.g., square wave, sine wave, sawtooth wave with a falling edge, sawtooth wave with a rising edge, etc.),
(vii) the quantity of photons being emitted to a user, which may depend on one or more of the above parameters, and/or
(viii) any combination of these parameters and/or other parameters.

In one embodiment, the sound emitter sub-system 150 may be configured to apply a predetermined auditory stimulus to the user. The predetermined auditory stimulus may be defined according to various parameters including:
(i) one or more predetermined beat frequencies (including any frequency and/or phase offset between the beat frequencies and the pulse frequencies emitted by the light emitter sub-system 140),
(ii) individual beat frequencies between different audio channels (e.g., left and right channels, such as to emit binaural beats),
(iii) audio frequencies of the one or more beat frequencies, including one or more individual audio frequencies for each predetermined beat frequency and/or audio channel,
(iv) one or more predetermined intensities or amplitudes of the beat frequencies, including one or more individual intensities for each predetermined beat frequency, audio channel, and/or audio frequency,
(v) one or more durations to emit each beat frequency, audio frequency, and/or intensity for each audio channel,
(vi) pulse waveform shape,
(vii) other audio emission (e.g., musical accompaniment to the beat(s)), and/or
(viii) any combination of these parameters and/or other parameters.

The controller sub-system 130 may control the emitter sub-system 120 including the light emitter sub-system 140 and/or the sound emitter sub-system 150, to control the attributes of light and/or sound stimuli to the user.

According to an embodiment, additional stimuli beyond light and auditory may be employed in the system.

### Light Emitter Sub-System

The light emitter sub-system 140 may include one or more lights to deliver light-based stimulus to the user. The one or more lights may be, for example, a micro-light emitting diode (micro-LED) or LED configured to controllably emit light according to the parameters described above, based on control by the controller sub-system 130.

In one embodiment, the lights of the light emitter sub-system 140 may be configured as single-wavelength or narrow-wavelength emitters (e.g., LEDs) distributed in a predetermined arrangement so as to direct light of specific wavelength(s) (or narrow wavelength bands) to a specific field/zone of vision of the user.

In one embodiment, the light emitters are distributed in a non-uniform manner along the field/zone of vision of a user. In one embodiment, light emitters (e.g., single-wavelength emitters) emitting ultraviolet and/or purple light wavelengths are distributed with greater concentration at regions corresponding to a peripheral field/zone of vision of the user, compared to regions corresponding to a central field/zone of vision. In one embodiment, light emitters (e.g., single-wavelength emitters) emitting ultraviolet and/or purple light wavelengths are only provided at regions corresponding to a peripheral field/zone of vision of the user, and are not provided at regions corresponding to a central field/zone of vision. In one embodiment, light emitters (e.g., single-wavelength emitters) emitting red and/or infrared light wavelengths are distributed with greater concentration at regions corresponding to a central field/zone of vision of the user, compared to regions corresponding to a peripheral field/zone of vision. In one embodiment, light emitters (e.g., single-wavelength emitters) emitting red and/or infrared light wavelengths are only provided at regions corresponding to a central field/zone of vision of the user, and are not provided at regions corresponding to a peripheral field/zone of vision. In one embodiment, the light emitters are arranged in a left-right symmetrical pattern.

In one embodiment, the light emitters (e.g., single-wavelength emitters) are grouped according to their emitted wavelengths. In one embodiment, the number of groups (i.e., the number of emitted single wavelengths or narrow wavelength bands) is greater than 3. In one embodiment, the number of groups is greater than 4. In one embodiment, the number of groups is in a range between 4 and 16. In one embodiment, the number of groups is in a range between 6 and 10. In one embodiment, the number of groups is 8.

In one embodiment, the light emitters within each group may all be identical to one another. In one embodiment, at least two light emitters within an individual group may differ from one another.

In one embodiment, the groups of single-wavelength emitters correspond to respective color channels controlled by the controller sub-system 130. In one embodiment, the light emitter sub-subsystem 140 may contain 192 LEDs, split into 8 color channels, where each color channel corresponds to a different peak wavelength and has 24 identical LEDs. In one embodiment, the light emitter sub-system 140 may include 24 LEDs in each color channel, which is split into 3 smaller “pixels” of 8 identical LEDs in series. In one embodiment, the 24 “pixels” (i.e., 8 channels of 3 pixels) for each color channel are driven by a pulse width modulation (PWM) constant-current sink LED driver with an internal oscillator. The PWM driver may provide PWM control to each pixel based on a grayscale value for an individual “frame” of an experience to be provided to the user. In one embodiment, the controller sub-system 130 may provide all 3 “pixels” within a given color with the same control information (e.g., without further splitting the pixels into smaller spatial zones). Of course, it will be appreciated that the device may include a different number of color channels, a different grouping of “pixels”, a different number of total light emitters per color channel, and/or other different characteristics than those exemplary characteristics described herein.

In one embodiment, the light emitters are selected to have a narrow spectral output and to collectively summarize the visible spectrum. In one embodiment where the number of color channels is 8, the corresponding channels of wavelengths or narrow wavelength bands may be:

| | | | Luminous | |
|---|---|---|---|---|
| | Wavelength | Viewing | Intensity | |
| Nominal color | (nm) | Angle (deg) | (mcd) | Number |
| Ultraviolet | 390-395 | 130 | — | 24 |
| Purple/UV 2 | 405-410 | 130 | — | 24 |
| Blue | 468 | 130 | 115 | 24 |
| Green | 515 | 140 | 430 | 24 |
| True Green | 530 | 120 | 350 | 24 |
| Red | 639 | 130 | 54 | 24 |
| Deep Red | 660 | 140 | 16 | 24 |
| Far Red/IR | 700 | 140 | 5 | 24 |

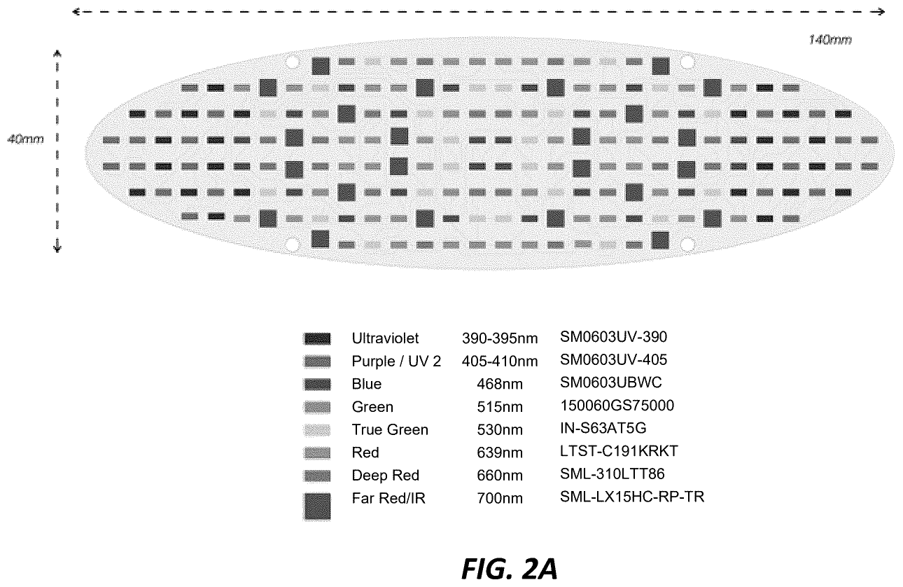

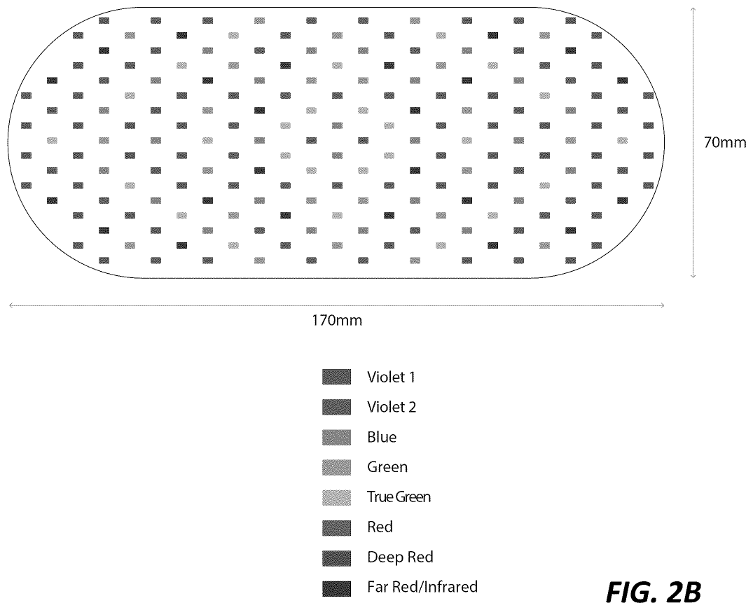

In one embodiment, single-wavelength emitters of the light emitter sub-system 140 may be arranged as illustrated in FIG. 2A. In one embodiment, where the number of color channels is 8, the corresponding

channels of wavelengths or narrow wavelength bands may be:

| | | | Luminous | |
|---|---|---|---|---|
| | Wavelength | Viewing | Intensity | |
| Nominal color | (nm) | Angle (deg) | (mcd) | Number |
| Violet 1 | 405 | 130 | — | 24 |
| Violet 2 | 415 | 150 | — | 24 |
| Blue | 468 | 130 | 115 | 24 |
| Green | 515 | 140 | 430 | 24 |
| True Green | 530 | 120 | 350 | 24 |
| Red | 639 | 130 | 54 | 24 |
| Deep Red | 660 | 140 | 16 | 24 |
| Far Red/IR | 700 | 140 | 5 | 24 |

In one embodiment, single-wavelength emitters of the light emitter sub-system 140 may be arranged as illustrated in FIG. 2B.

The arrangements of single-wavelength emitters described in FIGS. 2A and 2B may provide the capability to deliver light dosages of specific wavelengths to specific areas of a user's field of vision, while also providing a greater concentration of light emitters of particular wavelengths in certain regions (e.g., violet or ultraviolet wavelengths more concentrated at a peripheral region).

In one embodiment, at least a subset of the single wavelengths or narrow wavelength bands emitted by the light emitter sub-systems are beyond the visually perceptible range for humans. In one embodiment, the light emitter sub-system 120 and/or controller sub-system 130 are configured to emit the dosage(s) of light at least partially while the user's eyes are open. In one embodiment, the light emitter sub-system 120 and/or controller sub-system 130 are configured to emit the dosage(s) of light while the user's eyes are closed. In one embodiment, the light emitter sub-system 120 and/or controller sub-system 130 are configured to emit the dosage(s) of light only while the user's eyes are closed. In one embodiment, the emitted dosage(s) of light produce a conscious visual perception in the user, rather than biologically-active light that may not be perceived consciously such as light affecting melatonin suppression (melanoptic blue light to the eyes) or production of vitamin D by the skin exposed to UV.

### Sound Emitter Sub-System

According to an embodiment, the sound emitter sub-system 150 may include one or more speakers to deliver auditory stimulus to the user. The sound emitter sub-system 150 may alternatively or additionally include one or more interfaces (e.g., 3.5 mm audio jack, RCA or digital audio jacks, or Bluetooth) allowing the connection of peripheral audio components (e.g., headphones) for emitting the auditory stimulus to the user. For instance, wired or wireless headphones may be used for delivering binaural-beat auditory stimulus to the user. The sound emitter sub-system 150 may be configured to controllably emit audio according to the parameters described above, based on control by the controller sub-system 130.

### Controller Sub-System

According to an embodiment, the controller sub-system 130 may utilize a general-purpose computing device 400, as explained in more detail below. In one embodiment, the controller sub-system 130 stores pre-programmed experiences of stimulus to present to a user, such as synchronized control sequences for the light emitter sub-system 140 and/or sound emitter sub-system 150, and controls these sub-systems accordingly to present the experience (e.g., including the defined dosing of light) to the user. In one embodiment, the controller sub-system 130 is configured to receive and store defined experiences (and/or modify existing stored experiences) based on information from an external source (e.g., over a network, from a USB storage device, based on user input and/or control parameters, etc.).

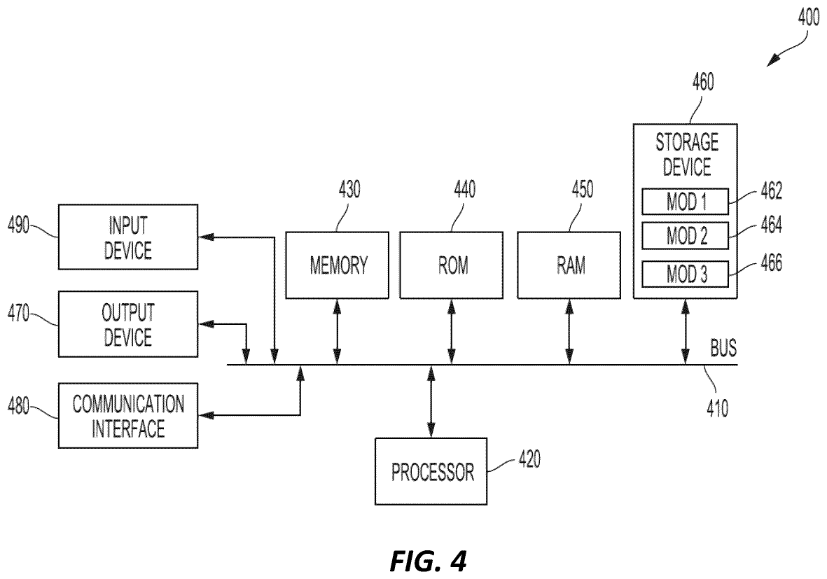

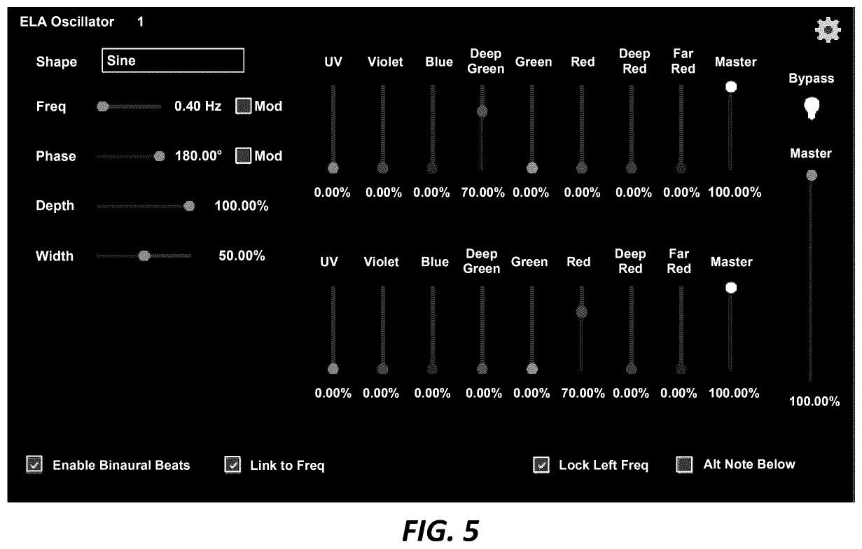

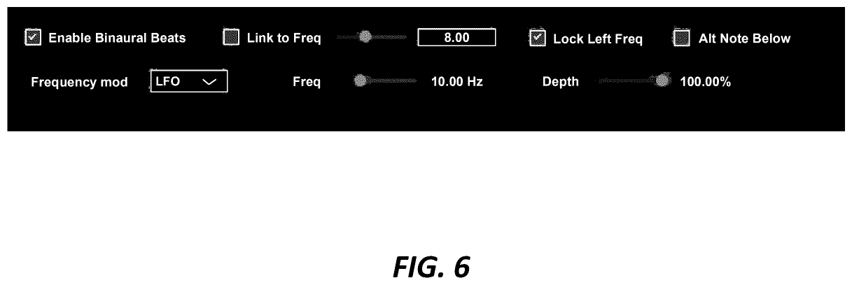

With reference to FIG. 4, an exemplary arrangement of the controller sub-system 130 described above includes a general-purpose computing device 400, including a processing unit (CPU or processor) 420 and a system bus 410 that couples various system components including the system memory 430 such as read-only memory (ROM) 440 and random access memory (RAM) 450 to the processor 420. The system 400 can include a cache of high-speed memory connected directly with, in close proximity to, or integrated as part of the processor 420. The system 400 copies data from the memory 430 and/or the storage device 460 to the cache for quick access by the processor 420. In this way, the cache provides a performance boost that avoids processor 420 delays while waiting for data. These and other modules can control or be configured to control the processor 420 to perform various actions. Other system memory 430 may be available for use as well. The memory 430 can include multiple different types of memory with different performance characteristics. It can be appreciated that the disclosure may operate on a computing device 400 with more than one processor 420 or on a group or cluster of computing devices networked together to provide greater processing capability. The processor 420 can include any general purpose processor and a hardware module or software module, such as module 1 462, module 2 464, and module 3 466 stored in storage device 460, configured to control the processor 420 as well as a special-purpose processor where software instructions are incorporated into the actual processor design. The processor 420 may essentially be a completely self-contained computing system, containing multiple cores or processors, a bus, memory controller, cache, etc. A multi-core processor may be symmetric or asymmetric.

The system bus 410 may be any of several types of bus structures including a memory bus or memory controller, a peripheral bus, and a local bus using any of a variety of bus architectures. A basic input/output (BIOS) stored in ROM 440 or the like, may provide the basic routine that helps to transfer information between elements within the computing device 400, such as during start-up. The computing device 400 further includes storage devices 460 such as a hard disk drive, a magnetic disk drive, an optical disk drive, tape drive or the like. The storage device 460 can include software modules 462, 464, 466 for controlling the processor 420. Other hardware or software modules are contemplated. The storage device 460 is connected to the system bus 410 by a drive interface. The drives and the associated computer-readable storage media provide nonvolatile storage of computer-readable instructions, data structures, program modules and other data for the computing device 400. In one aspect, a hardware module that performs a particular function includes the software component stored in a tangible computer-readable storage medium in connection with the necessary hardware components, such as the processor 420, bus 410, display 470, and so forth, to carry out the function. In another aspect, the system can use a processor and computer-readable storage medium to store instructions which, when executed by the processor, cause the processor to perform a method or other specific actions. The basic components and appropriate variations are contemplated depending on the type of device, such as whether the device 400 is a small, handheld computing device, a desktop computer, or a computer server.

Although the exemplary embodiment described herein employs the hard disk 460, other types of computer-readable media which can store data that are accessible by a computer, such as magnetic cassettes, flash memory cards, digital versatile disks, cartridges, random access memories (RAMs) 450, and read-only memory (ROM) 440, may also be used in the exemplary operating environment. Tangible computer-readable storage media, computer-readable storage devices, or computer-readable memory devices, expressly exclude media such as transitory waves, energy, carrier signals, electromagnetic waves, and signals per se.

To enable user interaction with the computing device 400, an input device 490 represents any number of input mechanisms, such as a microphone for speech, a touch-sensitive screen for gesture or graphical input, keyboard, mouse, motion input, speech and so forth. An output device 470 can also be one or more of a number of output mechanisms known to those of skill in the art. In some instances, multimodal systems enable a user to provide multiple types of input to communicate with the computing device 400. The communications interface 480 generally governs and manages the user input and system output. There is no restriction on operating on any particular hardware arrangement and therefore the basic features here may easily be substituted for improved hardware or firmware arrangements as they are developed.

### Methods of Treating Medical Indications

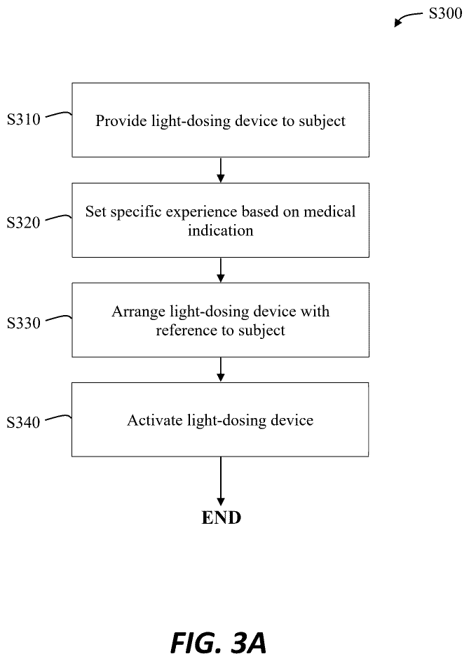

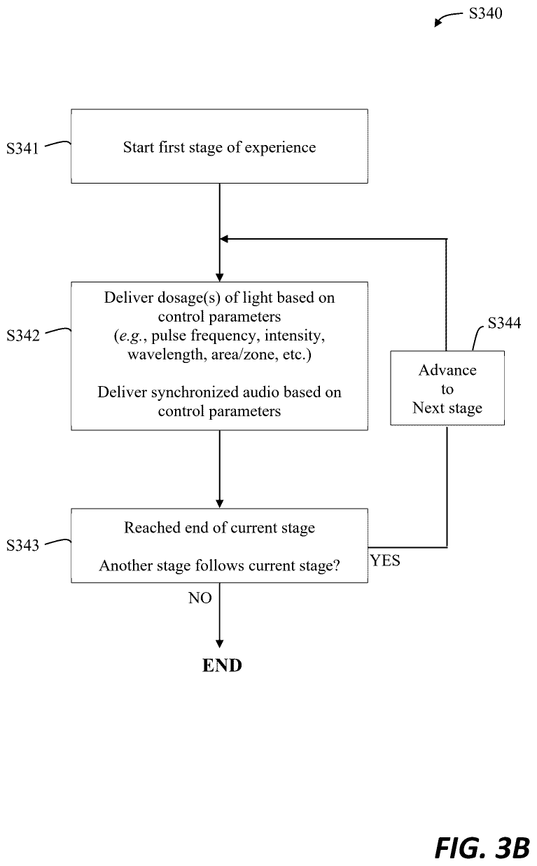

Various methods of treating medical indications according to the invention will now be described. FIG. 3A illustrates a first embodiment of a process S300 for treating medical indications in accordance with the invention. First, in step S310, the subject is provided with a light-dosing device (or system), such as those exemplary systems and devices described herein.

In step S320, a user controls the light-dosing device to set a specific light-dosing experience corresponding to a desired medical indication for treatment. For example, the light-dosing device may contain multiple programmed experiences, each corresponding to a specific medical indication for treatment. In one embodiment, the light-dosing device may contain multiple programmed experiences even for a single medical indication. The user performing the control in this step may be the subject him/herself, a medical professional, and/or any individual capable of operating the light-dosing device.

In step S330, the light-dosing device is physically arranged with reference to the subject. For instance, the device may be placed such that its light emitters face the subject's eyes. In one embodiment, the device may be physically attached to the subject. In another embodiment, the device may not be physically attached to the subject, but is retained by a retention mechanism such that it continuously faces the subject's eyes. In one embodiment, the device is placed proximate to the subject such that sound emitted from the device is perceptible by the subject. In one embodiment, peripheral audio devices (e.g., wired or wireless headphones) are physically and/or informationally coupled with the device.

In step S340, the light-dosing device is activated and administers the experience to the user. This experience may involve specific controlled and sequenced dosing of light delivered to the user's eyes based on specific control parameters, as described above. The experience may also involve specific controlled and sequenced emissions of audio information based on specific control parameters, as described above. The emissions of light and auditory stimulus may be synchronized with each other, according to the defined experience.

The experience may also involve modification of one or more parameters during the presentation of the experience. For instance, the experience may be divided into “stages”, where each successive stage involves different values of the various light and/or sound control parameters from the previous stage. As one example, the light pulse frequency may differ between different stages. As another example, the emitted light wavelengths may differ between different stages. It will be appreciated that any of the control parameters described herein may be varied between different stages. It will also be appreciated that any of the control parameters described herein may even be varied within the same stage. For instance, within a single stage, the pulse frequency may continuously alternate between two or more frequencies, may immediately change from a first frequency to a second frequency, or may ramp/sweep from a first frequency to a second frequency. The other parameters described herein may likewise vary within an individual stage and between different stages.

FIG. 3B illustrates additional steps within step S340 for presenting the experience to the user. In step S341, the controller sub-system 130 initiates the first stage of the selected experience. In step S342, the controller sub-system 130 controls the light emitter sub-system 140 to emit the defined dosage(s) of light, according to the stage of the experience. The controller sub-system 130 also controls the sound emitter sub-system 150 to emit auditory stimulus according to the stage of the experience, synchronized with the light emission.

The defined dosage(s) of light may include the definition of specific control parameters as described above, including but not limited to pulse frequency (or frequencies), intensity (or intensities), light wavelength(s), area(s)/zone(s), duration, pulse shape, and/or other control parameters. Likewise, the defined auditory stimulus may include the definition of specific control parameters as described above.

In step S343, the controller sub-system 130 determines that the end of the current stage has been reached, and determines whether another stage follows the current stage. If a subsequent stage exists in the experience, the method advances to step S344. If the current stage is the last stage in the experience, the method ends.

In step S344, the controller sub-system 130 advances to the next stage and then returns to step S342 to administer the next stage to the subject.

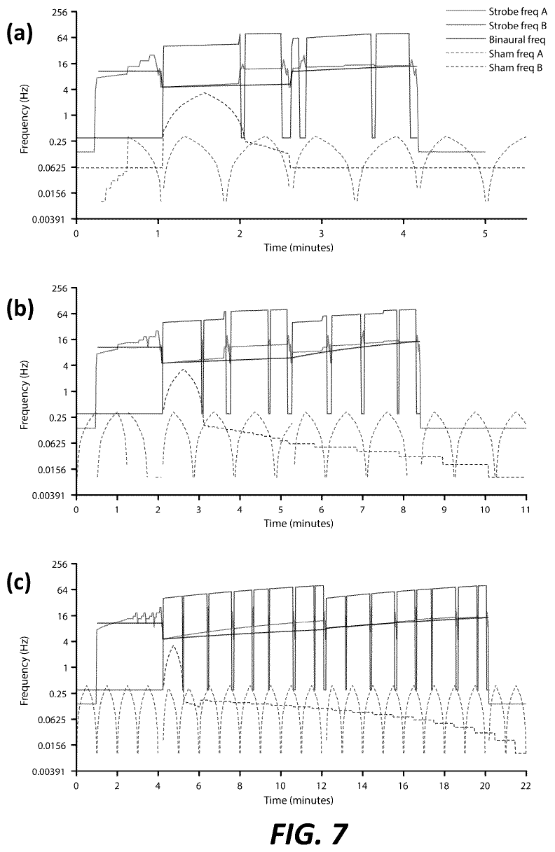

In an embodiment, process S300 may present a user experience that employs two or more light pulse frequencies (such as strobe frequencies). In one embodiment, the light dosing device may emit light to form a main strobe (“strobe A”) that varies from 0.14 to 25 Hz in pulse frequency, with a median frequency across time of 8-9 Hz (e.g., 8.8 Hz, 5.5 minute session; 8.5 Hz, 11 minute session; 9.6 Hz, 22 minute session). The light dosing device may additionally emit light to form a secondary strobe (“Strobe B”) that starts at 0.3 Hz in pulse frequency and flashes periodically 1-4 minutes into the experience (FIG. 7) with a frequency range of 40.1 to 79.4 Hz, with a median frequency across time (excluding the low frequency floor of 0.3 Hz at the beginning of each session) of 60-70 Hz (e.g., 68.5 Hz, 5.5 minute session; 60.0 Hz, 11 minute session; 59.6 Hz, 22 minute session). Additionally, the sound emitter sub-system 150 may emit binaural beats (4.5-14.5 Hz), and optionally incorporated with ambient music during each session. The frequency of the binaural beats may approximately (e.g., closely) follow the frequency of strobe A (FIG. 7). In one embodiment, the median binaural beat frequency across time may be 10.5 Hz (5.5 minute session), 8.1 Hz (11 minute session), and 9.3 Hz (22 minute session).

In an embodiment, process S300 may present a user experience according to the strobe and/or binaural frequency patterns set forth in FIG. 7(a), 7(b), or 7(c).

In an ne embodiment, the emitted main strobe may have a median frequency between 7-15 Hz, more preferably between 8-12 Hz, and even more preferably approximately 10 Hz.

The inventors have discovered that the controlled dosing of light may be effective to treat various medical indications. It will be appreciated that medical indications as described herein include not only physical and/or physiological ailments, but also human function(s) to be improved (e.g., cognitive abilities, alertness, etc.). The medical indications that may be treated/improved by such controlled light dosing include, but are not limited to (organized by general category and further divided into non-limiting consumer/clinical applications where applicable):

### Sleep Support

| Consumer | Clinical |
|---|---|
| Children Audio/Visual | Nighttime Urination & Rumination |
| Lullaby/Narrative | Issue Content |
| Poor Sleep & Insomnia Relief | Melatonin, DHEA, Serotonin |
| Content | Release Content |
| Bedtime Trance & Dream Content | Sub-Visual Circadian System |

### Meditation/Medical

| Consumer | Clinical |
|---|---|
| Narrative Intention & Prayer | Post Stroke Stimulation Content |
| Content | PTSD - Fear, Terror & Trauma |
| Mind-Body Integration, Kinesthetic | Resolution Content |
| Awareness, Synesthetic Integration | Acute Pain Reduction Content |
| Ecstatic Trance Content | Headache & Migraine Reduction |
| Awe, Wonder, Inspiration Content | Content |
| Sensory Vision Quest Content | Pre & Post Anesthesia Support |
| | Blood Pressure (e.g., hypertension) |
| | & Variable Heart Rate Regulation |
| | Content |
| | Fibromyalgia Symptom Relief |
| | Content |
| | Neuropathic Pain Reduction |
| | Content (Specifically for Spinal |
| | Injury) |
| | Parkinson Symptom Relief Content |
| | Sensory Integration Support |
| | Content |
| | Autonomic Hormone & Biomarker |
| | Regulation |
| | Hybrid Pharmacological/Audio- |
| | Visual Protocols |
| | Ritlan/Adderoll/Methylphenidate |
| | Reduction Protocols |
| | Hybrid Psychiatric Protocols for |
| | Mental Disorders |
| | Pain, headache, or migrane |

### Stress and Anxiety

| Consumer | Clinical |
|---|---|
| Stress & Anxiety Mitigation | Stress & Anxiety Mitigation |
| Stress Support Content for Terminal | Cortisol Reduction Content |
| Diagnosis | Content for Hospice |
| Stress Support for Chemotherapy | |
| Treatments | |
| Social & Situational Anxiety Relief | |
| Content | |
| Geriatric Boredom, Anxiety, | |
| Irritation | |
| Canine Barking Control Content | |

### Relaxation

Neck & Mandibular Tension Release Content
Cannabis/Ketamine/Psychedelic Support & Enhancement Content
Open & Closed Eye Content
Primal Eco-Nature Content (Land/Ocean/Sky/Cosmos/Jungle Gaia Series)

### Performance Enhancement

| Consumer | Clinical |
|---|---|
| Mood Shift & Improvement | Pre & Post Counselling, EMDR & |
| Content | Therapy Content |
| Compassion & Empathy Content | Autonomic Nervous System Self |
| Emotional Endurance & Tolerance | Regulation Content |
| Content | Dyslexia Mitigation Content |
| Irritability Improvement Content | Mood Disorder Support Content |
| Team Intention Integration & Flow | Mental Adaptation & Resilience |
| Content (Alpha Synchronization) | Content |
| Increase Working & Long-term | Cognitive Performance Content |
| Memory Content | Emotional Adaptation & Resilience |
| Creativity & Imagination | Content |
| Enhancement Content | Reactivity Reduction Content |
| Morning Wake-up Relax & Focus | Child/Adolescent Emotional |
| Content | Support Content |
| Social Bonding & Team Sync | Coping, Distress, Reactivity |
| Content | Reduction Support Content |
| Disturbance in the Field Content | Individual & Collective Grief, |
| (Rumination Reduction) | Sadness & Loss Support Content |
| Flow & Go With the Flow | |

### Cognitive Intervention

ADD/ADHD/OCD/ASD Spectrum Support
Endorphin & Dopamine Release Content
Alzheimer & Dementia Support Content
Attention-deficit/hyperactivity disorder

### Emotional Intervention

| Consumer | Clinical |
|---|---|
| Attachment & Separation Issues | Rage/Anger & Aggression |
| Child/Adolescent Emotional | Management Content |
| Support Content | Addiction or substance abuse |
| Post Gaming Autonomic | Management |
| Downshift Content | Opioid use reduction, either post |
| Coping, Distress, Reactivity | addiction or as support of reduced |
| Reduction Support Content | dosage in analgesia |
| Depression Support Content | Oxytocin Elevation Content |
| Morning Wake-up Relax & Focus | Attachment & Separation Issues |
| Content | Depression Support Content |
| Social Bonding & Team Sync | Suicidal Rumination Relief Content |
| Content | Reduction of obsessive/fixative |
| Disturbance in the Field | ideation |
| Content (Rumination Reduction) | PMS/Menopause Syndrome Relief |
| Flow & Go With the Flow | Content |
| Individual & Collective Grief, | Postpartum Blues & Depression |
| Sadness & Loss Support Content | Content |
| Childhood & Adult Shame & | Childhood & Adult Shame & |
| Negative Self Belief Support | Negative Self Belief Support |
| Content | Content |
| Autonomic Nervous System | Smoking Cessation Content |
| Support for VR Applications | News Cycle Addiction/Reaction |
| | Support Content |
| | Catharsis, Weeping & Crying |
| | Content |
| | Parasympathetic Nervous System |
| | Support Content |
| | Autonomic Nervous System |
| | Support for VR Applications |

The inventors have discovered that the light-dosing device may treat the medical indications by stimulating the user's central nervous system and metabolic systems and producing a desired brainwave state in the user.

### Efficacy Study

An efficacy study of the methods described herein will now be described.

### (1) Background

To better understand the approach to brain stimulation provided by the present invention, the inventors undertook a large study of periodic audiovisual stimulation (PAVS) in a large sample. While other studies have described the visual effects of stroboscopic stimulation, the neural effects are comparatively understudied, with just a handful of published studies which have largely focused on examining EEG frequency changes. The inventors thus sought to fill a gap in the literature by rigorously searching for entrainment effects, which have so far only been observed in a small, uncontrolled study.

Toward this end, the inventors studied the effects of stimulation in two active conditions with over 80 healthy participants each, one with multimodal synchronization between visual and auditory modalities (henceforth, “experimental stimulation”) and another lacking this component (henceforth, “sham stimulation”). Both groups were compared to a control condition with no stimulation in which a comparable number of participants were instead instructed to practice focused breathing meditation, which, like the active conditions, may also be considered a non-pharmacological altered state of consciousness. During stimulation, spontaneous EEG signals were recorded to test the hypothesis that neural oscillations are entrained by the frequency of PSS. In addition to neural entrainment effects, changes in mood, mindfulness, and other psychological variables were measured to test the secondary hypothesis that PAVS may carry psychological benefits that are able to be disassociated from its effects on visual perception. The inventors' findings offer clear evidence of neural entrainment induced by the visual component of PAVS implemented using the system 100.

### (2) Experimental Methods

### (a) Participants

A total of 273 participants, aged 19-79 (M=43.73; SD=15.58; 142 females) completed this study. Participants were not permitted to participate in this study if they had a history of epilepsy and/or seizures, migraines, photolight sensitivity, cataracts, corneal abrasions, keratitis, uveitis, hearing problems, or non-normal/non-corrected vision. Participants were also excluded if they were currently taking any photophobia-inducing medications or hearing-altering medications.

### (b) Materials

Bioperipherals were captured using the CGX Aim II Physiological Monitoring device (Cogniomics, Inc.) with ECG electrodes (Skintact, Inc.) and included Electromyography (EMG; 2 electrodes on the L/R base of the neck on the sternocleidomastoideole), Bio-Impedance-Based Respiration Rate (2 paddles with 2 electrodes each on the L/R pectoralis major), Heart Rate and Oxygen Saturation (finger clip), and Galvanic Skin Response (GSR; 2 electrodes on the palm of the non-dominant hand). Skin area was first cleansed with 91% isopropyl alcohol using a cotton pad.

EEG signal (500 Hz sampling rate) was acquired using a dual-amp 64-channel cap system (Brain Vision, LLC) connected to a 15.6″ 2021 Lenovo Ideapad. Measurements of the participant's head from their nasion to inion determined which cap size (54 cm, 56 cm, 58 cm, or 60 cm) was utilized. The cap was positioned on the participant's head such that channel FPz was at 10% of the distance from nasion to inion, midline channels were aligned, and the velcro chin strap was taught, but comfortable. Nuprep skin prep gel (Weaver and Co.) was used to exfoliate the scalp through electrodes before applying Neurospec abrasive electrolyte-gel (EasyCap, Inc.). All powered devices, with the exception of the stroboscopic device, were unplugged prior to the experiential portion of the experiment to help prevent the impact of line noise on the EEG data.

The stroboscopic device (INTO, Inc.) was an initial prototype of a phosphene generation device that uses an array of 8 color frequencies among 192 LEDs to output light through a 31% opacity diffuser. The LEDs were programmed to pulse at specific frequencies and the dynamic, time-varying patterns were paired with a pre-recorded stereo audio track.

Briefly, the experimental condition was designed to encourage a state of relaxation. It includes a light composition and an atmospheric auditory composition complimented with binaural beats. The placebo condition is an asynchronous series of pulsing light frequencies that modulate the 8 LED frequencies at irregular intervals. The intensity of the asynchronous light is identical to the light levels in the study protocol, therefore matching the lux output of the study protocol. The placebo audio is identical to the Experimental condition apart from the Binaural beat rhythms which are absent from the audio. The control condition included a simple breath-focused, eyes-closed meditation exercise, matched in duration to the experimental and placebo conditions.

Wired earbuds were provided to the participants to place in their ears. The device was positioned 5 inches away from the participant's nose using a desk mount swivel. All participants sat in a powered recliner chair regardless of group assignment and were instructed to adjust the leg and back positions to their comfort liking. All window shades were lowered prior to the start of each experiential portion of the experiment. Participants were instructed to keep their eyes closed throughout the duration of the light stimulation as the experience was intended to output light onto the eyelids.

Experiential compositions were triggered using Ableton Live 10 via a Python3 Controller, Pylive (https://github.com/ideoforms/pylive) on a 13.3″ 2020 Macbook Air. Lab Streaming Layer with LabRecorder (https://github.com/labstreaminglayer) was utilized to temporally synchronize EEG, peripheral, and experimental time series (e.g. pre-experience rest ended, Ableton experience started) within an XDF file format.

Randomization was conducted using a single-site validated block randomization model (Castor EDC) with gender as a randomization strata across 9 groups (3 experiences×3duration periods). Both the participants and experimenters were blind as to group assignment until after the first resting state period.

### (c) Procedure

Neither the participant nor the experimenter was aware of which experiential group the participant would be assigned to at this time. Immediately afterward, participants completed a suite of questionnaires and brief computer tasks for approximately 20 minutes. Participants were outfitted with the EEG cap, bio-peripherals, and earbuds (regardless of group) while seated in a recliner chair. Participants were then instructed to close their eyes and relax, trying their best not to fall asleep, for 5 minutes. Afterward, the experimental script would reveal the randomized group assignment to the participant and experimenter for the first time. Participants were assigned to either the Audiovisual Experience 1 (Experimental), Audiovisual Experience 2 (Sham), or Control group with a sub-group of either 5.5, 11, or 22 minutes, for a total of 9 groups.

If assigned to an audiovisual experience, participants had the device positioned in front of their closed eyes before the experimental script triggered the launch of the experience. Subjects were told they could easily swivel the mounting arm and exit the experience at any time if they so desired. If assigned to the meditation group, participants were read instructions for a breath-focused awareness meditation before engaging in the meditation in the same seated position as the other groups. Following this experiential period, participants were able to open their eyes briefly before engaging in a second period of 5-minute closed-eye rest. Afterwards, the EEG and bio peripherals were removed and participants were permitted to use the restroom to rinse their hair. Following this cleanup, participants completed a post-experience behavioral assay before being compensated and dismissed from the study.

### (3) EEG Methods

All analyses were split into 2 phases:
Phase 1: Pre-experience resting-state vs Post-experience resting state
Phase 2: On-line Experience

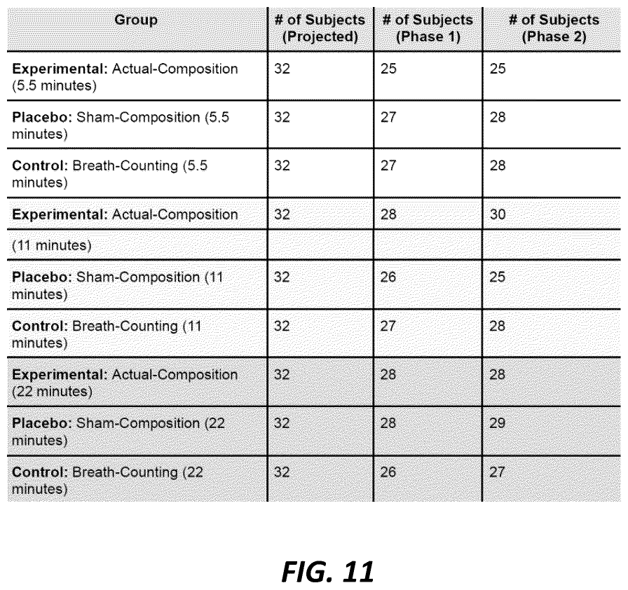

Exclusions were assessed based on testing protocol abnormalities provided by INTO and further outliers were identified from signal quality, missing data, or frequency band power statistics. All subjects identified as outliers were removed from group statistics and the number of subjects in each group for each phase of the analyses is listed in FIG. 11.

### (a) Pre-Processing

The data were subjected to a standard preprocessing chain in NeuroPype™ which removes and/or repairs data from corrupted electrodes and signal artifacts (drift, line noise, and high-variance artifacts including blinks, movement, and muscle) and prepares the data for subsequent analysis. This includes the following stages: 1) Inferring channel locations from their 10-20 labels; 2) Removal of channels that have no location (e.g., trigger channel, ExG); 3) FIR high-pass filter with a transition band between 0.1 and 0.5 Hz; 4) Removal of bad channels using a correlation and high-frequency noise criterion; 5) Removal of high-amplitude artifacts using Artifact Subspace Reconstruction with an artifact threshold of 10 s.d.; 6) FIR low-pass filter with a transition band between 45 and 50 Hz; 7) Spherical-spline interpolation of removed channels; 8) Electrode re-referencing to Common Average Reference; 9) Removal of residual high-artifact time windows using a variance criterion.

### (b) Power-Spectra and Frequency Bands

EEG power spectral density was estimated from each time window (2 seconds) using the Multitaper method, 1/f normalized, and converted to dB. The normalized PSD was further averaged within frequency bands to yield bandpower estimates for the following frequency bands: delta=1-4 Hz, theta=4-8 Hz, alpha=8-12 Hz, beta=12-32 Hz, and gamma=32-40 Hz.

### (c) Experiential Composition Correlations (Entrainment)

Frequency band power correlations with the photostrobic light and audio frequencies were computed for each EEG channel and source. The following steps were used to calculate the correlation coefficient of each feature: EEG power spectra used a longer time window epoch (4 sec as opposed to 2 sec) in order to create a narrower frequency bin without overlap into neighboring frequencies. EEG spectrum frequency bins are exactly 1 Hz and the provided (from the event markers) photostrobic/audio frequencies were thereby rounded to the nearest 1 Hz. For each of the 1 Hz bins of each of the photostrobic/audio frequency types a square wave over time was created to indicate when the given frequency was presented to the subject (high wave) and set to 0 (low) when it was not present. Each of the generated square waves were correlated with the same EEG spectral frequency bin over the entire ELA time course to compute a correlation coefficient (Pearson R) per bin. Since the Placebo and Control groups did not use the same photostrobic frequencies and timings as the Experimental group, those were handled by using the same generated square waves over time from the Experimental group (matching the correct dosage group) and running the same correlation steps. For each separate channel/source all the R values were averaged across all the frequency bins for each of the 3 ELA correlation types, such that for the entire session each channel/source has 1 mean R value per frequency correlation type. The group stats (ANOVAs) used those session mean R values such that the standard main effects of Group and Dose plus the Group*Dose interaction were computed for each of those 3 frequency correlation types (Frequency A, Frequency B, Audio).

### (d) Statistical Analyses

For individual sessions, Phase 1 analyses compared pre vs post resting state EEG spectral power and physiology metrics. Each session segment (pre, post) was treated as a within-subjects factor and an independent 2 sample t-test was used to test for significant differences (corrected for multiple comparisons using False Discovery Rate, Benjamini Hochberg 1995) for all metrics. Phase 2 analysis compared each of 6 session segments (pre-idle, part1, part2, part3a, part3, and post-idle) and additionally the entire “full experience” (the aggregate of all 6 segments) for each of the EEG and physiology metrics. There were no statistical tests used at the individual session level for Phase 2. For group level statistics, the inventors analyzed the effects for Group and Dose factors on spectral brain responses for selected channels and cortical sources as well as physiology metrics using the following 3-level full factorial design (32):

$$y = Group + Dose + Group * Dose$$

This design was computed with an unbalanced (Type III sum of squares), two-way mass-univariate ANOVA, corrected for multiple comparisons, but individual channels or sources were considered independent and not corrected. Post-hoc pairwise comparisons used Tukey HSD to account for family-wise error.

Both Phase 1 and Phase 2 analyses used the two-way ANOVA design on each dependent EEG and physiology metric variables for each of the independent session segments (Phase 1: pre, post; Phase 2: pre-idle, part1, part2, part3a, part3b, post-idle, full experience). Additionally, in order to analyze the effect of the different ELA stages over time for Phase 2, a second statistical analysis was conducted using a mixed model (between-subjects and within-subjects) repeated measure ANOVA using the following design:

$$y = Group + Stage + Group * Stage$$

In this case, the Dose factor was eliminated from the model as this analysis was run independently for each of the 3 Dose levels (5.5, 11, and 22 min). The inventors modeled differences in pre versus post stimulation EEG measures (power in each frequency band and LZC), as well as pre versus during stimulation EEG measures, using two-way ANOVAs with group and duration as independent variables. In order to account for multiple comparisons (one model fit for each EEG channel), spatial cluster permutation statistics were used to identify significant clusters of channels for each EEG measure. This was done by thresholding the F-values from each predictor (GROUP, DURATION, and GROUP×DURATION) in each model according to the corresponding critical value at P=0.01 to identify spatial clusters with large effects. The inventors then randomly permuted the data labels 10,000 times and fit models for each channel and permutation. This allowed construction of a null distribution of cluster sizes for each predictor and EEG measure. The clusters in the original data were then evaluated using empirical P-values computed from the null distribution of cluster sizes across permutations.

### (4) Results

Over 250 adult subjects without photosensitive contraindications participated in the experiment. Of these participants, the inventors retained usable EEG data recording during the experience of each group from N=248 participants. Recordings were analyzed from participants in each of three groups: 1) experimental stimulation (N=81), 2) sham stimulation (N=83), and 3) a breath-focused meditation control condition (N=84). Additionally, participants were assigned to one of three session durations independently of the group they were assigned to: 5.5 minutes (N=83), 11 minutes (N=82), and 22 minutes (N=83).

Stimulation frequencies varied throughout the PAVS experience in both experimental stimulation and the sham stimulation groups (FIG. 7). During the experimental stimulation, the frequency of the main strobe (“strobe A”) varied from 0.14 to 25 Hz, with a median frequency across time of 8-9 Hz (8.8 Hz, 5.5 minute session; 8.5 Hz, 11 minute session; 9.6 Hz, 22 minute session). Additionally, a secondary strobe (“strobe B”) was incorporated which began at 0.3 Hz and then started to flash periodically 1-4 minutes into the experience (FIG. 7) with a frequency range of 40.1 to 79.4 Hz; its median frequency across time (excluding the low frequency floor of 0.3 Hz at the beginning of each session) was 60-70 Hz (68.5 Hz, 5.5 minute session; 60.0 Hz, 11 minute session; 59.6 Hz, 22 minute session). Lastly, binaural beats (4.5 Hz-14.5 Hz) were incorporated with ambient music during each session to add an auditory dimension to the stimulation. The frequency of the binaural beats closely followed the frequency of strobe A (FIG. 7). When present, the median binaural beat frequency across time was 10.5 Hz (5.5 minute session), 8.1 Hz (11 minute session), and 9.3 Hz (22 minute session). During the sham stimulation, much lower stimulation frequencies were used which were expected to elicit far weaker effects. The frequency of strobe A varied from 0.01 to 0.38 Hz (excluding periods of constant intensity, i.e., 0 Hz stimulation), with a median frequency of 0.15 Hz (5.5 minute session), 0.16 Hz (11 minute session), or 0.19 Hz (22 minute session). Similarly, the frequency of strobe B varied from 0.01 to 3.25 Hz (excluding periods of 0 Hz stimulation), with a median frequency of 0.06 Hz (5.5 minute session), 0.05 Hz (11 minute session), or 1.0 Hz (22 minute session). Binaural beats were absent from the sham stimulation.

To quantify neural entertainment, the inventors correlated the binary presence/absence of a strobe at a given frequency taken from the experimental stimulation condition with spectral EEG power at the corresponding frequency. Correlations were computed by rounding strobe or binaural frequencies to the nearest 1 Hz bin. For each frequency bin, a square wave was created across time such indicating periods when the frequency is active in the stimulation. Each square wave was correlated with a corresponding time series of EEG spectral power at the same frequency, and this step was repeated separately for each EEG channel. Finally, correlation coefficients were averaged across frequency bins to yield an overall quantification of neural entrainment. Note that entrainment was always referenced to the experimental stimulation frequencies regardless of which group participants were assigned to, as the stimulation frequencies present in the sham stimulation were generally below the highpass filter cutoff frequency and no stimulation was present in the meditation control group. In this way, each comparison group (sham and control) served to demonstrate the extent of false positive entrainment which could be expected due to chance.

In what follows, all statistics were calculated using an unbalanced (Type III sum of squares), two-way mass-univariate ANOVA following a 3×3 Design: Group+Dose+Group: Dose. P-values were adjusted for testing across multiple channels using false discovery rates applied separately to each ANOVA.

### (a) Stroboscopic Stimulation Produces Widespread Neural Entrainment

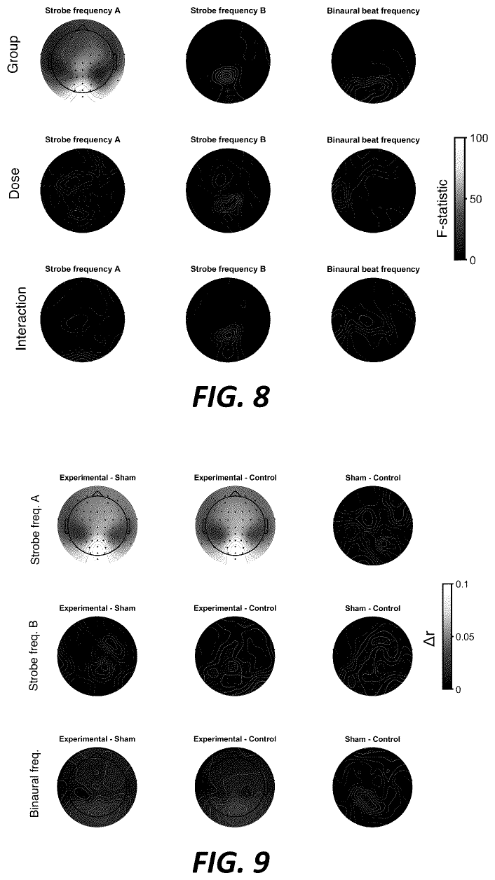

The inventors found a highly significant main effect of group at all EEG channels for entrainment by strobe A (F>15, P<0.001, all channels). Posthocs tests revealed that this effect was driven by the experimental versus sham contrast (P=0.0036, all channels) and the experimental versus control contrast (P=0.0036, all channels). Although this neural entrainment effect is widespread, the largest entrainment effects were detected over visual areas, fitting with the stimulus modality, and lowest over temporoparietal channels (see FIGS. 8 and 9). No posthoc tests were significant for the sham versus control contrast. Dose and Group×Dose interaction effects were not significant for any channels (FIG. 8).

Although the inventors attempted to measure entrainment effects from strobe B, this proved somewhat impractical as the median strobe frequency (60-70 Hz) was above the lowpass filter cutoff frequency. The inventors declined to adjust filter settings, as EEG frequencies above 45 Hz are substantially contaminated by muscle artifacts and electrical line noise in scalp recordings. Nonetheless, the inventors attempted to measure high frequency entrainment despite these challenges and found several posterior electrodes with a significant main effect of group at the frequency of strobe B (CPz, P3, P2, CP1, POZ, Oz, Iz, P<0.05). This result suggests focal entrainment of posterior areas (e.g., visual cortices). Posthocs tests revealed that three of these channels were significant in the contrast of experimental stimulation with the meditation control (CPz, CP1, Iz, P<0.05), but no channels were significant in the contrast of experimental stimulation with sham stimulation.

### (b) Evidence of a Photic Driving Response

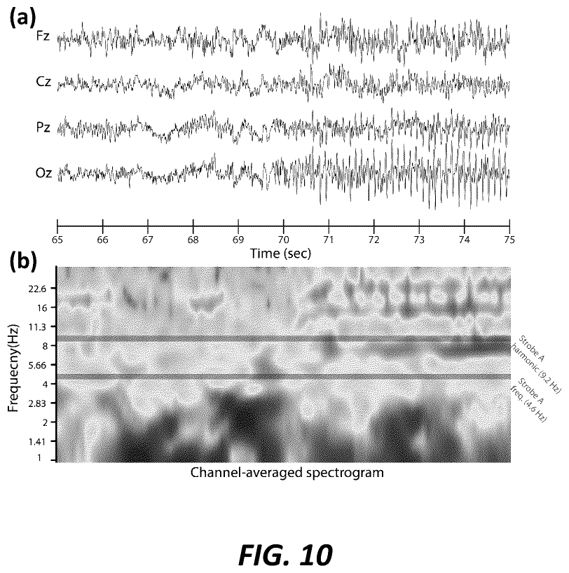

In addition to these quantitative results, the inventors also observed qualitative evidence in EEG waveforms of strong entrainment manifesting as a very well-defined yet benign photic driving response in at least one participant (FIG. 10(a)), which was independently reviewed by two neurologists. A Morlet wavelet transform was applied to all channels and the channel-averaged spectrogram was plotted (FIG. 10(b)), revealing a burst of spectral energy near the first harmonic of the strobe A frequency (4.6 Hz), as is common in the photic driving response. Note that because the photic driving response appears non-sinusoidal and was convolved with a Gaussian-windowed sinusoid (i.e., a Morlet wavelet), some spectral leakage and frequency shift may be present, potentially explaining why the response does not occur exactly at 9.2 Hz.

Use of language herein such as “at least one of X, Y, and Z,” “at least one of X, Y, or Z,” “at least one or more of X, Y, and Z,” “at least one or more of X, Y, or Z,” “at least one or more of X, Y, and/or Z,” or “at least one of X, Y, and/or Z,” are intended to be inclusive of both a single item (just X, or just Y, or just Z) and multiple items (i.e., {X and Y}, {X and Z}, {Y and Z}, or {X, Y, and Z}). “At least one of” is not intended to convey a requirement that each possible item must be present.

Although the foregoing description is directed to the embodiments of the invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

We claim:

1. A method of treating one or more medical indications using a device that includes at least one controller and a light emitter module, comprising:
controlling the light emitter module to emit first pulsed light starting at a first pulse frequency in a direction of a user's eyes; and
controlling the light emitter module to, while emitting the first pulsed light, emit second pulsed light at a second pulse frequency in the direction of the user's eyes.

2. The method of claim 1, wherein the first pulse frequency is in a range between 0.14-25 Hz.

3. The method of claim 1, wherein the second pulse frequency is in a range between 0.3-79.4 Hz.

4. The method of claim 1, wherein the controlling of the light emitter module includes emitting the light when the user's eyes are closed.

5. The method of claim 1, further comprising controlling the light emitter module to change the light emission of the first pulsed light from the first pulse frequency to a third pulse frequency.

6. The method of claim 1, wherein the first pulse frequency and the third pulse frequency are both is in a range between 0.14-25 Hz.

7. The method of claim 1, wherein a median frequency of the first pulsed light during an operation of the device is between 8-9 Hz.

8. The method of claim 7, wherein a median frequency of the second pulsed light during an operation of the device is between 60-70 Hz.

9. The method of claim 1, further comprising:
controlling an audio emitter to emit sound starting at a third pulse frequency.

10. The method of claim 9, wherein the pulse frequency of the emitted sound approximately follows the pulse frequency of the first pulsed light during operation of the device.

11. A system comprising:
at least one controller; and
a light emitter module,
wherein the controller is configured to control the light emitter module to:
emit first pulsed light starting at a first pulse frequency in a direction of a user's eyes, and
while emitting the first pulsed light, emit second pulsed light at a second pulse frequency in the direction of the user's eyes.

12. The system of claim 11, wherein the first pulse frequency is in a range between 7-15 Hz.

13. The system of claim 11, wherein the second pulse frequency is in a range between 0.3-79.4 Hz.

14. The system of claim 11, wherein the controller is configured to control the light emitter module to emit light when the user's eyes are closed.

15. The system of claim 11, further the controller is configured to control the light emitter module to change the light emission from the first pulse frequency to a third pulse frequency.

16. The system of claim 11, wherein the first pulse frequency and the third pulse frequency are both is in a range between 0.14-25 Hz.

17. The system of claim 11, wherein a median frequency of the first pulsed light during an operation of the device is between 8-9 Hz.

18. The system of claim 17, wherein a median frequency of the second pulsed light during an operation of the device is between 60-70 Hz.

19. The system of claim 11, wherein the controller is configured to control an audio emitter to emit sound starting at a third pulse frequency.

20. The system of claim 19, wherein the pulse frequency of the emitted sound approximately follows the pulse frequency of the first pulsed light during operation of the device.

21. A system comprising:
at least one controller; and
a light emitter module that includes a plurality of light-emitting diodes, each light-emitting diode emitting single-wavelength or narrow-wavelength-band light to a specific field of vision of a user, wherein at least one of the light-emitting diodes emits a different wavelength or wavelength band than another one of the light-emitting diodes,
wherein the controller is configured to control the light emitter module to:
emit first pulsed light starting at a first pulse frequency in a direction of a user's eyes,
while emitting the first pulsed light at the first pulse frequency, emit second pulsed light at a second pulse frequency in the direction of the user's eyes, and
change the light emission of the first pulsed light from the first pulse frequency to a third pulse frequency.

22. The system of claim 21, wherein a median frequency of the first pulsed light during an operation of the device is between 8-9 Hz.

23. The system of claim 22, wherein a median frequency of the second pulsed light during an operation of the device is between 60-70 Hz.
